SEQUENCE LISTING (3 sequences)

SEQ ID NO: 1 - protein, 716 aa, Influenza A virus
  N-terminal domain (1)..(196)
  Hinge (197)..(257)
  C-terminal domain (258)..(716)
MEDFVRQCFNPMIVELAEKAMKEYGEDLKIETNKFAAICTHLEVCFMYSDFHFIDEQGESIVVELGDPNALLKHRFEIIEGRDRTIAWTVINSICNTTGAEKPKFLPDLYDYKKNRFIEIGVTRREVHIYYLEKANKIKSEKTHIHIFSFTGEEMATKADYTLDEESRARIKTRLFTIRQEMASRGLWDSFRQSERGEETIEERFEITGTMRKLADQSLPPNFSSLENFRAYVDGFEPNGYIEGKLSQMSKEVNARIEPFLKSTPRPLRLPDGPPCSQRSKFLLMDALKLSIEDPSHEGEGIPLYDAIKCMRTFFGWKEPNVVKPHEKGINPNYLLSWKQVLAELQDIENEEKIPRTKNMKKTSQLKWALGENMAPEKVDFDDCKDVGDLKQYDSDEPELRSLASWIQNEFNKACELTDSSWIELDEIGEDAAPIEHIASMRRNYFTAEVSHCRATEYIMKGVYINTALLNASCAAMDDFQLIPMISKCRTKEGRRKTNLYGFIIKGRSHLRNDTDVVNFVSMEFSLTDPRLEPHKWEKYCVLEVGDMLLRSAIGHVSRPMFLYVRTNGTSKIKMKWGMEMRRCLLQSLQQIESMIEAESSVKEKDMTKEFFENKSETWPVGESPKGVEEGSIGKVCRTLLAKSVFNSLYASPQLEGFSAESRKLLLIVQALRDNLEPGTFDLGGLYEAIEECLINDPWVLLNASWFNSFLTHALR

SEQ ID NO: 2 - protein, 726 aa, Influenza B virus
  N-terminal domain (1)..(195)
  Hinge (196)..(254)
  C-terminal domain (255)..(726)
MDTFITRNFQTTIIQKAKNTMAEFSEDPELQPAMLFNICVHLEVCYVISDMNFLDEEGKTYTALEGQGKEQNLRPQYEVIEGMPRNIAWMVQRSLAQEHGIETPRYLADLFDYKTKRFIEVGITKGLADDYFWKKKEKLGNSMELMIFSYNQDYSLSNESSLDEEGKGRVLSRLTELQAELSLKNLWQVLIGEEDIEKGIDFKLGQTISKLRDISVPAGFSNFEGMRSYIDNIDPKGAIERNLARMSPLVSVTPKKLKWEDLRPIGPHIYNHELPEVPYNAFLLMSDELGLANMTEGKSKKPKILAKECLEKYSTLRDQTDPILIMKSEKANENFLWKLWRDCVNTISNEETSNELQKTNYAKWATGDGLTYQKIMKEVAIDDETMYQEEPKIPNKCRVAAWVQTEMNLLSTLTSKRALDLPEIGPDVAPVEHVGSERRKYFVNEINYCKASTVMMKYVLFHTSLLNESNASMGKYKVIPITNRVVNEKGESFDMLYGLAVKGQSHLRGDTDVVTVVTFEFSSTDPRVDSGKWPKYTVFRIGSLFVSGREKSVYLYCRVNGTNKIQMKWGMEARRCLLQSMQQMEAIVEQESSIQGYDMTKACFKGDRVNSPKTFSIGTQEGKLVKGSFGKALRVIFTKCLMHYVFGNAQLEGFSAESRRLLLLIQALKDRKGPWVFDLEGMYSGIEECISNNPWVIQSAYWFNEWLGFEKEGSKVLESVDEIMDE

SEQ ID NO: 3 - protein, 709 aa, Influenza C virus
  N-terminal domain (1)..(178)
  Hinge (179)..(238)
  C-terminal domain (239)..(709)
MSKTFAEIAEAFLEPEAVRIAKEAVEEYGDHERKIIQIGIHFQVCCMFCDEYLSTNGSDRFVLIEGRKRGTAVSLQNELCKSYDLEPLPFLCDIFDREEKQFVEIGITRKADDSYFQSKFGKLGNSCKIFVFSYDGRLDKNCEGPMEEQKLRIFSFLATAADFLRKENMFNEIFLPDNEETIIEMKKGKTFLKLRDESVPLPFQTYEQMKDYCEKFKGNPRELASKVSQMQSNIKLPIKHYEQNKFRQIRLPKGPMAPYTHKFLMEEAWMFTKISDPERSRAGEILIDFFKKGNLSAIRPKDKPLQGKYPIHYKNLWNQIKAAIADRTMVINENDHSEFLGGIGRASKKIPEVSLTQDVITTEGLKQSENKLPEPRSFPKWFNAEWMWAIKDSDLTGWVPMAEYPPADNELEDYAEHLNKTMEGVLQGTNCAREMGKCILTVGALMTECRLFPGKIKVVPIYARSKERKSMQEGLPVPSEMDCLFGICVKSKSHLNKDDGMYTIITFEFSIREPNLEKHQKYTVFEAGHTTVRMKKGESVIGREVPLYLYCRTTALSKIKNDWLSKARRCFITTMDTVETICLRESAKAEENLVEKTLNEKQMWIGKKNGELIAQPLREALRVQLVQQFYFCIYNDSQLEGFCNEQKKILMALEGDKKNKSSFGFNPEGLLEKIEECLINNPMCLFMAQRLNELVIEASKRGAKFFKID

(19) United States
(12) Patent Application Publication
(10) Pub. No.: US 2016/0076006 A1
(43) Pub. Date: Mar. 17, 2016

(54) THERMOSENSITIVE MUTANTS OF INFLUENZA VIRUSES

(71) Applicant: INSTITUT NATIONAL DE LA RECHERCHE AGRONOMIQUE, Paris (FR)

(72) Inventor: Bernard DELMAS, Bourge-la reine (FR)

(73) Assignee: INSTITUT NATIONAL DE LA RECHERCHE AGRONOMIQUE, Paris (FR)

(21) Appl. No.: 14/784,827

(22) PCT Filed: Apr. 18, 2014
(86) PCT No.: PCT/IB2014/060840

§ 371 Date: Oct. 15, 2015

(30) Foreign Application Priority Data
Apr. 18, 2013 (FR) 1353543

(51) Int. Cl. C12N 7/00; A61K 39/145

(52) U.S. Cl. CPC C12N 7/00; A61K 39/145; C12N 2760/16121; C12N 2760/16151; C12N 2760/16134; A61K 2039/525

(57) ABSTRACT

The invention relates to thermosensitive mutants of influenza viruses, containing at least one mutation localised in a flexible region of a protein of said virus, such as especially the region of the sub-unit PA of the viral polymerase corresponding to the region 197-225 of the sub-unit PA of influenza A. These viruses can be especially used for preparing attenuated living vaccines.

The present invention relates to thermosensitive mutants of influenza viruses.

Influenza viruses, which are the agents of flu, are among the main respiratory pathogens of human beings and animals. There are three types of influenza virus: A, B and C, the first two, and in particular influenza A, being the most important from the point of view of human pathology, by virtue of its frequency and the potential seriousness of the associated pathological conditions.

Influenza viruses belong to the family Orthomyxoviridae. They have a segmented negative single-stranded RNA genome composed of eight segments in influenza A and B, and of seven segments in influenza C. In influenza virus types A and B, segments 1, 2 and 3 encode, respectively, the three subunits of the RNA-dependent RNA polymerase: the two acid subunits PB2 and PB1, and the acid subunit PA; segment 4 encodes one of the two surface glycoproteins, hemagglutinin IIA; segment 5 encodes the nucleoprotein NP, which combines with the viral RNAs to form the nucleocapsid; segment 6 encodes the other surface glycoprotein, neuraminidase NA; segment 7 encodes the matrix proteins M1 and M2, and segment 8 encodes the non-structural proteins NS1 and NS2.

In the type C viruses, there is just one surface glycoprotein: hemagglutinin esterase (HE), which is encoded by segment 4, and which replaces the hemagglutinin and the neuraminidase.

The prevention of flu is based mainly on vaccination. The type of vaccine most commonly sold is the inactivated vaccine, based on viruses cultivated on embryonated hen's eggs, then chemically inactivated. More recently live attenuated vaccines have emerged, constructed from donor viral strains having mutations which attenuate their pathogenicity. The vaccine strains are viruses referred to as “reassortant”, the six internal genes of which are those from the donor strain, and the two HA and NA genes of which are those from the viral strain with respect to which it is desired to induce protection.

Live attenuated vaccines, which are administered intranasally, have the advantage of simulating the natural infection, and of making it possible to induce both a local and a systemic immune response.

These live attenuated vaccines were initially developed from “cold-adapted” donor strains obtained by a series of passages in culture at 25° C. It was observed that the mutants resulting therefrom exhibited a particular phenotype, combining cold adaptation (ca), thermosensitivity (ts) and attenuation of virulence (att).

Thermosensitivity is a very advantageous characteristic for obtaining live attenuated vaccines. Indeed, thermosensitive mutants are capable of multiplying normally at temperatures corresponding to those encountered in the upper respiratory tract of infected animals (generally from 30 to 33° C. depending on the temperature of the inhaled air), and are thus enabling the initiation of a mucosal and serum immune response; on the other hand, their thermosensitivity limits their replication in the lower respiratory pathways and the lungs. By way of examples, mention will be made of the ca strain A/Ann Arbor/6/60, or the ca strain B/Ann Arbor/1/66, which are used as donor strains in the FluMist™ attenuated vaccine: the first of these strains replicates normally up to 37° C., but its replication is inhibited at 39° C., contrary to the wild-type influenza A strains, which replicate efficiently up to 40-41° C.; the second replicates normally up to 33° C., but, contrary to the wild-type influenza B strains, its replication capacity decreases at high temperatures, and is inhibited at 37° C.

Reverse genetic approaches have made it possible to identify genetic determinants involved in the thermosensitivity of these strains, and to transfer them to other strains in order to reproduce the thermosensitive phenotype. Thus, it has been observed that the ca strain B/Ann Arbor/1/66 contains eight amino acid substitutions, located in PB2, NP, PA and M1, and it has been shown that three of them (V114A and P410H on NP and V431M on PA) are involved in its thermosensitivity, and that two additional mutations in M1 confer thereon its attenuated nature; the introduction of these mutations in the wild-type strain B/Yamanashi/166/98 has also made it possible to transfer, to said strain, the same phenotypic characteristics (Hoffmann et al., Journal of Virology, 79, 11014-21, 2005 and PCT international application WO 03/091401). Likewise, the ca strain A/Ann Arbor/6/60 contains five amino acid substitutions (N34G on the nucleoprotein NP, N265S on PB2, K391E, E581G, and A661T on PB1) involved in its thermosensitive nature and/or in the attenuation of its pathogenicity (Jin et al., Virology, 306, 18-24, 2003); the introduction of these mutations into the genome of the strain A/Puerto Rico/8/34 has made it possible to confer, on said strain, the thermosensitive phenotype of the ca strain A/Ann Arbor/6/60 (Jin et al., Journal of Virology, 78, 995-98, 2004); the same mutations, introduced into the genome of the strain A/New York/1682/2009 have also enabled the acquisition, by said strain, of a thermosensitive nature, which has been further accentuated by the introduction of supplementary mutations (P112S, N556D, Y658H) into PB2 (Zhou et al., Vaccine, 30, 3691-702, 2012).

These thermosensitive mutants also constitute tools that are very useful for studying the viral cycle. For example, the study of a thermosensitive mutant in the non-structural protein NS1 has made it possible to show that this protein is involved in a late viral morphogenesis event (Garaigorta et al., J Virol, 79, 15246-57, 2005). More recently, the study of a thermosensitive mutation in the nucleoprotein NP has made it possible to identify the role played by NP in the formation of infectious particles (Noton et al., J Virol, 83, 562-71, 2009).

The inventors have undertaken the identification of other mutations which may confer a thermosensitive phenotype in influenza viruses, and have more particularly addressed the viral polymerase, and in particular the PA subunit.

The RNA-dependent RNA polymerase of influenza viruses is a heterotrimer consisting of an acidic subunit, PA, and of two basic subunits, PB1 and PB2 (for review, cf. Boivin et al., J Biol Chem, 285, 28411-7, 2010). These subunits are encoded by the longest three segments of the viral genome. In combination with the NP and NS2 proteins (Robb et al., J Gen Virol, 90, 1398-407, 2009), these three subunits ensure the transcription and replication of the viral genome.

PB1, which is encoded by segment 2, constitutes the catalytic subunit of the polymerase, and contains the characteristic motifs of RNA-dependent RNA polymerases (Poch et al., Embo J, 8, 3867-74, 1989; Muller et al., J Gen Virol, 75 (Pt 6), 1345-52, 1994). Two minor translation products of segment 2 have also been described; one resulting from an internal start codon and producing an N-terminally truncated form of PB1, and the other resulting from a reading-frame shift and producing a form called PB1-F2 which is associated with the virulence of certain strains (Wise et al., J Virol, 83, 8021-31, 2009; Chen et al., Nat Med, 7, 1306-12, 2001).

The PB2 subunit, encoded by segment 1, is involved in the binding to the cap located at the 5′ end of the messenger RNAs of the host cell (Blaas et al., Nucleic Acids Res, 10, 4803-12, 1982; Ulmanen et al., Proc Natl Acad. Sci U S A, 78, 7355-9, 1981). This cap will subsequently be cleaved in order to be used as a primer for initiating viral RNA synthesis.

The PA subunit, encoded by segment 3, has a length of 716 amino acids in influenza A, 726 amino acids in influenza B, and 709 amino acids in influenza C; it contains two structurally well-defined domains: an N-terminal domain (amino acids 1-196 in influenza A, 1-195 in influenza B, and 1-178 in influenza C) and a C-terminal domain (amino acids 258-716 in influenza A, 255-726 in influenza B, and 239-709 in influenza C). These two domains are separated by a 60 amino acid hinge region.

The N-terminal domain of the PA subunit is related to the PD-(D/E)XK nuclease family (Dias et al., Nature, 458, 914-8, 2009; Yuan et al., Nature, 458, 909-13, 2009) and has an endonuclease activity involved in cleavage of the host-cell mRNA cap. The C-terminal domain binds for the first 15 residues of the PB1 subunit, and it is supposedly involved in viral RNA transcription: it has been observed that a substitution in this domain (His→Ala in position 510 of PA) strongly inhibits this transcription, without affecting viral replication (Fodor et al., J Virol, 76, 8989-9001, 2002).

Segment 3 contains a second open reading frame accessible by ribosomal shifting (Jagger et al., Science, 337, 199-204, 2012). The resulting translation product, called PA-X, contains the endonuclease domain, followed by a C-terminal domain of 61 residues, encoded by the ORF X, and involved in the repression of the expression of the host cell genes. The ORF X overlaps with a large part of the reading frame encoding the hinge separating the endonuclease domain from the PB1-binding domain of PA.

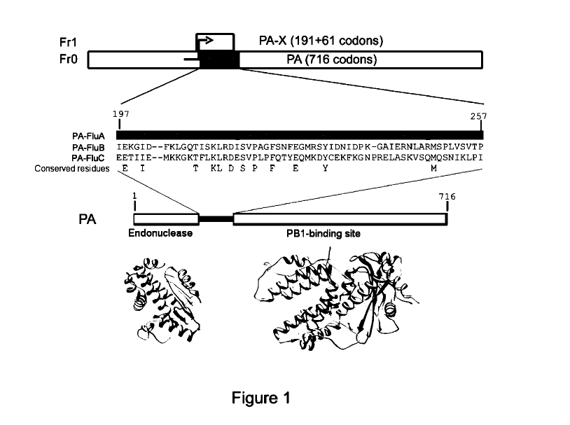

The structure of the PA subunit is represented in FIG. 1:

Top of the figure: reading frames of segment 3: frame 0 corresponds to the PA protein, and frame 1 to PA-X;

Middle of the figure: alignment of the amino acid sequences of the hinge regions of the PA proteins of the influenza A, B, and C viruses; the conserved residues are listed;

Bottom of the figure: three-dimensional structure of the PA protein, showing the endonuclease domain and the PB1-binding domain, connected by the hinge region.

By way of examples, the complete polypeptide sequences of the PA subunits of influenza A virus (strain A/WSN/1933(H1N1); GenBank ACF54605.1), influenza virus B (strain B/Ann Arbor/1/1986; GenBank ABF21265.1), and influenza virus C (strain C/Ann Arbor/1/50; GenBank YP_{—}089654.1) are respectively represented in the appended sequence listing under SEQ ID Nos: 1, 2 and 3. The sequences of the hinge regions represented in FIG. 1 correspond respectively to amino acids 197-257 of SEQ ID No.: 1, to amino acids 196-254 of SEQ ID No.: 2, and to amino acids 179-238 of SEQ ID No.: 3.

At the current time, most thermosensitive-phenotype-conferring influenza virus polymerase mutations have been identified in the PB1 and PB2 subunits. With regard to the PA subunit, in addition to the V431M mutation, located in the PB1-binding domain in the strain B/Ann Arbor/1/66, two other mutations conferring a thermosensitive phenotype have recently been identified in influenza A strains: one (L226P) is located in the hinge region (Kawaguchi et al., J Virol, 79, 732-44, 2005), and the other (F35S) in the endonuclease domain (Zhang et al., Virology Journal, 9, 97, 2012).

The inventors have now performed various synthetic mutations in the influenza genes, all located in a flexible region of the corresponding protein, such as, for example, region 197-225 in influenza A (corresponding to region 196-222 of the PA subunit in influenza B, and to region 179-205 of the PA subunit in influenza C), and conferring a thermosensitive phenotype on the viral strains which carry them.

A synthetic mutation, as opposed to a natural or induced mutation, is defined herein as a mutation not previously identified in a thermosensitive strain of influenza viruses.

A flexible region of a protein is defined as a region which does not have a stable secondary structure. This region may be defined on the basis of the atomic structure of the protein, when said structure has been characterized. For example, the structure of the NP protein of the influenza virus (strain A/WSN/1933(H1N1)) is described in Ye et al., Nature, 444, 1078-1082, 2006. When the atomic structure of the protein is not known, the secondary structure of the protein or of a region of this protein can be determined using conventional techniques known to those skilled in the art, such as, for example, circular dichroism analysis.

A thermosensitive mutant of an influenza virus is defined herein as a mutant of which the maximum permissive temperature (i.e. the temperature up to which it can replicate normally) is lower than that of the wild-type virus from which it is derived. Below the maximum permissive temperature, this thermosensitive mutant has a replication similar to that of the wild-type virus from which it is derived; above the maximum permissive temperature, its replication is reduced by at least two times, preferably at least five times, advantageously at least ten times, and entirely preferably at least 100 times compared with the wild-type virus from which it is derived.

Consequently, a subject of the present invention is a method for preparing a thermosensitive mutant of an influenza virus, characterized in that at least one synthetic mutation is introduced into a region of a gene of said virus corresponding to a flexible region of the protein encoded by said gene, and said mutation generating a thermosensitive mutant of said virus.

In accordance with the method of the invention, one or more mutations are introduced into one or more regions of one or more genes of said influenza virus; preferably, said mutations induce the substitution or the deletion of an amino acid.

In accordance with the method of the invention, said mutation(s) is (are) introduced into any viral protein (PA, PB1, PB2, HA, NA, HE, NP, M1, M2, NS1, NS2).

According to one advantageous embodiment of said method, said mutation(s) is (are) located in a gene encoding a protein associated with the replicative complex, i.e. the PA, PB1, PB2 or NP protein, preferably the PA or NP protein.

The mutation(s) in the PA gene is (are) preferably located in the region of the gene encoding the PA protein corresponding to region 197-257 of the PA protein of the influenza A virus. Preferably, said mutation(s) in the gene encoding the PA subunit of the polymerase of said virus is (are) located in the region of said gene encoding the region of the PA subunit corresponding to region 197-225 of the PA subunit of influenza A. Even more preferably, said mutation(s) is (are) chosen from:
a mutation resulting in the substitution of the amino acid corresponding to position 210 of the PA subunit of influenza A, by an amino acid other than a threonine, preferably by an amino acid chosen from proline, phenylalanine, histidine, tryptophan, tyrosine, glycine, isoleucine, leucine, valine, aspartic acid, glutamic acid, lysine and arginine, and entirely preferably by a proline;
a mutation resulting in the substitution of the amino acid corresponding to position 213 of the PA subunit of influenza A, by an amino acid other than an arginine or a lysine, preferably by an amino acid chosen from proline, phenylalanine, histidine, tryptophan, tyrosine, glycine, isoleucine, leucine, valine, aspartic acid and glutamic acid, and entirely preferably by a proline;
a mutation resulting in the substitution of the amino acid corresponding to position 216 of the PA subunit of influenza A, by an amino acid other than an asparagine or an aspartic acid, preferably by an amino acid chosen from proline, phenylalanine, histidine, tryptophan, tyrosine, glycine, isoleucine, leucine, valine, lysine and arginine, and entirely preferably by a proline;
a mutation resulting in the substitution of the amino acid corresponding to position 219 of the PA subunit of influenza A, by an amino acid other than a leucine or a valine, preferably by an amino acid chosen from alanine, proline, cysteine, asparagine, glutamine, serine, threonine and tyrosine, and entirely preferably by an alanine or a proline;
a mutation resulting in the substitution of the amino acid corresponding to position 221 of the PA subunit of influenza A, by an amino acid other than a proline in the case of influenza A, an alanine in the case of influenza B, and a leucine in the case of influenza C; preferably, this amino acid will be chosen from alanine, glycine, isoleucine, leucine, methionine, phenylalanine, valine, tryptophan, cysteine, asparagine, glutamine, serine, threonine and tyrosine in the case of influenza A; from proline, phenylalanine, glycine, histidine, tryptophan, tyrosine, glycine, isoleucine, leucine, valine, cysteine, asparagine, glutamine, serine, threonine and tyrosine in the case of influenza B; and from alanine, proline, cysteine, asparagine, glutamine, serine, threonine, phenylalanine, glycine, histidine, tryptophan and tyrosine in the case of influenza C; entirely preferably, it will be an alanine in the case of influenza A, a proline in the case of influenza B, and an alanine or a proline in the case of influenza C;
a mutation resulting in the substitution of the amino acid corresponding to position 222 of the PA subunit of influenza A, by an amino acid other than an asparagine in the case of influenza A, a glycine in the case of influenza B, and a proline in the case of influenza C; preferably, this amino acid will be chosen from proline, alanine, glycine, isoleucine, leucine, methionine, phenylalanine, valine, tryptophan, aspartic acid, glutamic acid, histidine, lysine and arginine in the case of influenza A; from proline, alanine, cysteine, asparagine, glutamine, serine, threonine, isoleucine, leucine, valine, phenylalanine, histidine, tryptophan and tyrosine in the case of influenza B; and from alanine, glycine, isoleucine, leucine, methionine, phenylalanine, valine, tryptophan, cysteine, asparagine, glutamine, serine, threonine and tyrosine in the case of influenza C; entirely preferably, it will be an alanine or a proline in the case of influenza A and of influenza B, and an alanine in the case of influenza C;
a mutation resulting in the substitution of the amino acid corresponding to position 223 of the PA subunit of influenza A, by an amino acid other than a phenylalanine, preferably by an amino acid chosen from alanine, proline, cysteine, asparagine, glutamine, serine, threonine, tyrosine, glycine, isoleucine, leucine and valine, and entirely preferably by an alanine or a proline;
a mutation resulting in the substitution of the amino acid corresponding to position 225 of the PA subunit of influenza A, by an amino acid other than a serine in the case of influenza A, an asparagine in the case of influenza B, and a threonine in the case of influenza C, preferably by an amino acid chosen from alanine, proline, aspartic acid, glutamic acid, histidine, lysine, arginine, glycine, isoleucine, leucine, methionine, phenylalanine, valine and tryptophan in the case of influenza A; from alanine, proline, aspartic acid, glutamic acid, histidine, lysine, arginine, glycine, isoleucine, leucine, methionine, phenylalanine, valine and tryptophan in the case of influenza B; and from alanine, proline, phenylalanine, histidine, tryptophan, tyrosine, aspartic acid, glutamic acid, lysine and arginine in the case of influenza C, and entirely preferably by an alanine or a proline.

The amino acids corresponding to positions 207, 208, 209, 210, 213, 216, 219, 221, 222, 223 and 225 of the PA subunit of the influenza A virus polymerase are respectively located at positions 204, 205, 206, 207, 210, 213, 216, 218, 219, 220 and 222 of the PA subunit of the influenza B virus polymerase, and at positions 187, 188, 189, 190, 193, 196, 199, 201, 202, 203 and 205 of the influenza C virus polymerase.

Mutations that are particularly preferred in the PA gene are those which induce the substitution of at least one of the amino acids corresponding to positions 210, 216, 219, 221, 222, 223 and 225 and entirely preferably of at least one of the amino acids corresponding to positions 216 and 219 of said PA subunit in influenza A.

Other mutations that are particularly preferred in the PA gene are those which induce the deletion of at least one of the amino acids corresponding to positions 207, 208, 209 or 210 of the PA protein of the influenza A virus.

The mutation(s) in the NP gene is (are) preferably located in the region of the gene encoding the NP protein corresponding to region 73 to 92, 203 to 212, 230 and 231, 297 to 301, 429 to 436 or 490 to 498 of the NP protein of the influenza A virus. Preferably, said mutations are located in one of said regions of the gene encoding the NP protein of an influenza A virus or in the region of the gene encoding the NP protein of an influenza B virus corresponding to region 203 to 212 of the NP protein of the influenza A virus.

Said positions in the NP protein of the influenza A virus (A/Wilson-Smith/1933(H1N1)) are indicated with reference to the SWISSPROT P15682/GenBank M30746.1 sequence.

Preferred mutations in the NP gene are those which induce the deletion or the substitution of at least one of the amino acids corresponding to position 205, 206, 209, 210 or 211 of the NP protein of the influenza A virus. The substitution is, for example, the replacement of the amino acid in position 206 or 210 by an alanine or a proline, preferably a proline. Alternatively, the substitution is the replacement of the amino acid in position 205, 209 or 211 by a proline.

The amino acids corresponding to positions 73 to 92, 203 to 212, 230 and 231, 297 to 301, 429 to 436 and 490 to 498 of the NP protein of the influenza A virus are respectively located at positions 125 to 147, 264 to 270, 288 and 289, 454 to 457, 485 to 492, and 553 to 560 of the NP protein of influenza B virus, and to positions 66 to 87, 207 to 216, 234 and 235, 409 to 412, 440 to 447 and 530 to 537 of the NP protein of the influenza C virus.

In order to limit the frequency of revertants, several substitutions or a deletion are advantageously introduced into said virus, such as, for example, the substitutions D216P and T210P or else one of the deletions d207, d208, d209 or d210 in PA. Alternatively, the mutation is chosen so that, in order to obtain the substitution of the desired codon, several nucleotides of said codon are changed. It is also possible to introduce several mutations of which one is a deletion in order to decrease the frequency of revertants.

One or more of the mutations in accordance with the invention described above may optionally be combined with other mutations, in order to further improve the phenotypic properties of the mutated virus in the context of a vaccine use, for example in order to increase its thermosensitivity and/or to increase the attenuation of its virulence. These mutations can be located in other regions of the PA subunit, and/or in one or more of the other viral proteins, in particular PB1, PB2, NP or NS1.

It is, for example, possible to introduce a mutation in accordance with the invention into the genome of an influenza A or B strain, preferably an attenuated strain, such as the A/Ann Arbor/6/60 or B/Ann Arbor/1/66 strain mentioned above.

However, the mutations in accordance with the invention generally cannot be combined with certain additional “back” mutations which have been identified by the inventors. This is because these back mutations compensate for the phenotypic effects of the mutations in accordance with the invention, and therefore result in total or partial restoration of a wild-type phenotype.

These are the following mutations:
a combination of mutations identified on the thermosensitive virus having the substitution T210P: one of these mutations located on codon 377 of the PA gene induces the substitution of a glutamic acid to lysine in the PB1-interacting domain of PA, and the other, located on codon 20 of the PA gene, includes the substitution of an alanine to threonine in the endonuclease of PA;
a back mutation also identified on the thermosensitive virus having the substitution T210P is located on codon 287 of the PB1 gene, and induces the substitution of an arginine to methionine; in the thermosensitive mutant D216P, this substitution R287M induces only a partial reversion to the wild-type phenotype.

The method in accordance with the invention can be carried out using the conventional techniques for producing recombinant influenza viruses by reverse genetics. These techniques are well known in themselves to those skilled in the art (cf., for example, Neumann & Kawaoka, Virology, 287, 243-50, 2001; Fodor et al., Journal of Virology, 73, 9679-82, 1999; Jackson et al., Journal of General Virology, 92, 1-17, 2011; Neumann et al., Influenza Virus: Methods and Protocols, 865, 193-206, 2012; Zhou & Wentworth, Methods Mol Biol, 865, 175-92, 2012; LeGoff et al., PLoS One., 2012, 7(8)e37095).

A subject of the present invention is also the mutant recombinant influenza viruses obtained by means of a method in accordance with the invention. These viruses are characterized in that they contain at least one mutation in accordance with the invention as defined above, in particular a mutation in the gene encoding the PA subunit of the polymerase.

Mutant recombinant influenza viruses in accordance with the invention can be used in particular as donor strains for obtaining live attenuated flu vaccines. The reassortant viruses obtained from these donor strains, and also the live attenuated vaccines containing these reassortants viruses, are also part of the subject of the present invention.

The present invention will be understood more clearly by means of the additional description which follows, which refers to examples illustrating the obtaining of thermosensitive influenza viruses in accordance with the invention.

### EXAMPLES

### Materials and Methods:

### Viral Strains:

The viral strains A/WSN/1933(H1N1) and A(H1N1)pdm09 were used as support to produce the mutations in PA.

Cell cultures: The 293 T cells were cultured in DMEM medium (Dulbecco's modified Eagle medium) supplemented with 10% of fetal calf serum. The MDCK (Madin-Darby canine kidney) cells were cultured in minimum essential medium containing 5% of fetal calf serum (FCS).

### Viral Protein Mutagenesis:

The plasmids mutated in the PA or NP gene were constructed using the QuickChange mutagenesis kit (Stratagene) to mutate the codons of the flexible regions of the corresponding protein. The mutations introduced were confirmed by sequencing the plasmids generated.

### Plasmids Used:

The pPolI-WSN-NA-luciferase plasmid produces an RNA similar to the viral RNA of segment 6, in which the region encoding neuraminidase is replaced by the sequence encoding firefly luciferase.

### Recombined Virus Generation:

The viruses used were generated by reverse genetics. The mutants derived from the A/WSN/1933(H1N1) strain were generated from plasmids carrying the eight genomic segments of the A/WSN/1933(H1N1) strain, and four plasmids encoding the four proteins of the replication complex (PA, PB1, PB2 and NP), using the protocol described by Fodor et al., Journal of Virology, 73, 9679-82, 1999. These plasmids were used to transfect cocultures of 293T and MDCK cells. 48 hours after transfection, the viruses were harvested, and used to inoculate MDCK cells for the production of the viral stock. The mutants derived from the A(H1N1)pdm09 strain were generated using the system of reverse genetics of the influenza virus A(H1N1)pdm09 described in LeGoff et al., PLoS One, 2012, 7(8): e37095. The PA genes of the viruses obtained were sequenced to confirm the presence of the desired mutations and the absence of undesired mutations.

### Viral Replication Tests:

MDCK cells were infected at 33° C., 37° C. and 39.5° C., with the wild-type WSN virus or each of the PA mutants, at a multiplicity of infection (MOI) of 0.01.

At various times after infection, the cultured supernators were harvested, and the viral titer was determined on MDCK cell plates.

### Luciferase Activity Tests:

293T cells grown in wells of P96 plates were transfected with plasmids deriving from the pcDNA3 plasmid (Invitrogen) and expressing the (wild-type or mutant) PB1, PB2, NP and PA proteins and the pPolI-WSN-NA-luciferase plasmid. The amounts of plasmid for the transfections were 50 μg/well for pcDNA3-PA and mutated pcDNA3-PA, pcDNA3-PB1 and pcDNA3-PB2, 85 μg/well for pcDNA-NP and 133 μg/well for pPolI-WSN-NA-luciferase. The pRSV-β-Gal plasmid (Promega) was cotransfected (50 μg/well) and the assaying of the β-Gal activity was used as an internal control and standardization of the transfection efficiency. By way of negative control, the 293T cells were transfected with the same plasmids, with the exception of the one expressing PA. After transfection, the cells were incubated at 33° C. or 39.5° C. for 48 h, then lysed, and the luciferase activity in the lysate was measured in the presence of luciferin, a substrate for luciferase, using a Tecan luminometer, according to the manufacturer's instructions.

### Pathogenicity Tests:

C57B1/6 mice (n=10) received, intranasally, successive ten-fold dilutions of A/WSN/1933 virus or of the D216P and L219P mutant viruses (from 10⁶ to 10² plaque-forming units/mouse, in 50 pl of PBS buffer), and were weighed daily. The mice having a weight loss greater than 25% were considered to be dead, and were euthanized.

### Infections and Blood Samples Following Infection

C57Bl/6 mice (n=5) anesthetized with a mixture of ketamine and xylazine (1 and 0.2 mg per mouse, respectively) were intranasally infected with 50 μl of PBS containing 10⁴ to 10² plaque-forming units/mouse of A/WSN/1933 virus or of the D216P and L219P mutant viruses. On day 18, the mice were euthanized and blood samples were taken from them.

### Assaying of Anti-Influenza Antibodies

The antibodies specific for the influenza A virus (total Ig) were tested by ELISA in the individual sera of mice infected with the mutant viruses. The influenza A virus antigen (200 ng per well in 100 μl of 0.1 M carbonate-bicarbonate buffer, pH 9.5) were deposited in microtitration plates (Immulon 2HB, ThermoLabsystems), then the plates were incubated overnight at 4° C. The plates were washed five times with PBS-0.05% Tween® 20 between each step of the test. After the antigen adsorption step, the residual protein-binding sites were saturated with PBS-T-FCS buffer (5% FCS in PBS-0.05% Tween® 20) for one hour at 37° C. The samples were diluted successively three-fold in PBS-T-FCS buffer, beginning with a 1:30 dilution, then dispensed into the plates which were incubated for 2 h at 37° C. The antigen-bound antibodies were detected using a goat anti-mouse IgH+L antibody conjugated to horseradish peroxidase (P.A.R.I.S.; 1 ng/ml) incubated for one hour at 37° C. The TMB substrate (Kirkegaard & Perry Laboratories Inc.) was added for ten minutes, then the reaction was stopped by adding 1M phosphoric acid. The absorbance was measured at 450 nm with an ELISA plate reader (Dynex, MRX visualization). The results were expressed by the limiting antibody titers calculated by regression analysis of the dilution as a function of A450 (regression curve y=(b+cx)/(1+ax)). The limiting titers were determined by the highest dilution giving an absorbance two times greater than that of the negative control.

### Example 1

### Generation and Characterization of Mutants

Mutations were produced in the hinge region of the PA protein by selecting the amino acids conserved between the influenza A, B and C viruses, and also those located in proximity to these residues. In total, 25 amino acid positions were selected. Two types of substitutions were performed: substitutions by a proline residue in order to disrupt the potential structuring of this domain (except for positions 220 and 221 where there is already a proline), or substitutions by an alanine residue, because of its methyl functional group, which is of low hindrance and is chemically inert. Since the reading frame of PA and the ORF-X overlap, several of the substitutions performed in the hinge of PA also generated mutations in PA-X. In this case, in order to determine whether the phenotype observed resulted from a mutation in PA or in PA-X, modifications were introduced into the ribosomal reading frame shift motif, from UUU CGU (codons 191 and 192) to UUC AGA in order to reduce the ribosomal phase shift efficiency and, consequently, the expression of PA-X. The mutants containing these modifications are denoted hereinafter 191FS. In addition, deletions of a single amino acid residue were carried out in positions 205, 206, 207, 208, 209, 210, 211 and 212 of PA mutants d205, d206, d207, d208, d209, d210, d211 and d212.

Thirty mutant viruses, with or without modifications of the ribosomal reading frame shift motif, were thus generated. The remaining three mutants defined by substitutions could not be produced, suggesting that the corresponding mutations (L214P, S218P, and E237P) are lethal for the virus. The deletion mutants with a deletion in positions 205, 206, 211 and 212 could not be produced, suggesting that the corresponding mutations (d205, d206, d211 and d212) are lethal for the virus.

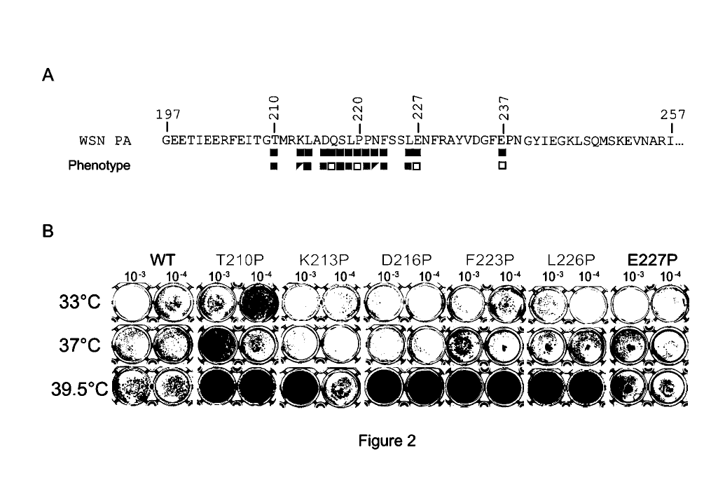

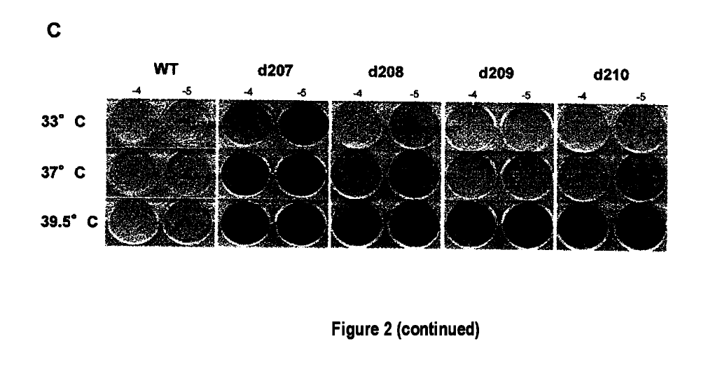

The substitution mutations produced are represented in FIG. 2A. The amino acid sequence of the hinge domain of PA of influenza A, and the residues conserved between the A, B, and C viruses are indicated. The positions chosen for the substitution mutations are indicated by dark gray squares. The substitutions conferring a thermosensitive phenotype are indicated by a light gray square. The substitutions which do not allow the production of viruses are indicated by a black square, and the substitutions not conferring a thermosensitive phenotype on the viruses produced are indicated by a white square.

The replication properties in cell cultures of the mutant viruses were examined by MDCK cell plaque titration.

The results obtained with the T210P, K213P, D216P, F223P, L226P and E227P mutants are illustrated by FIG. 2B. Several of the mutants tested (T210P, K213P, D216P, F223P, S225P and L226P) show a marked thermosensitivity. Although they do not lyse MDCK cells, or only weakly lyse them, at 39.5° C., they have a lytic efficiency comparable to that of the WSN wild-type virus at 33° C. and 37° C. Only the E227P mutant has a lytic efficiency comparable to that of the wild-type virus at the three temperatures tested.

The results obtained with the d207, d208, d209 and d210 mutants are illustrated by FIG. 2C. The d207, d208, d209 and d210 mutants show a marked thermosensitivity. These deletion mutants have a lytic activity that is more marked at 33° C. than at 37° C. and than at 39.5° C.

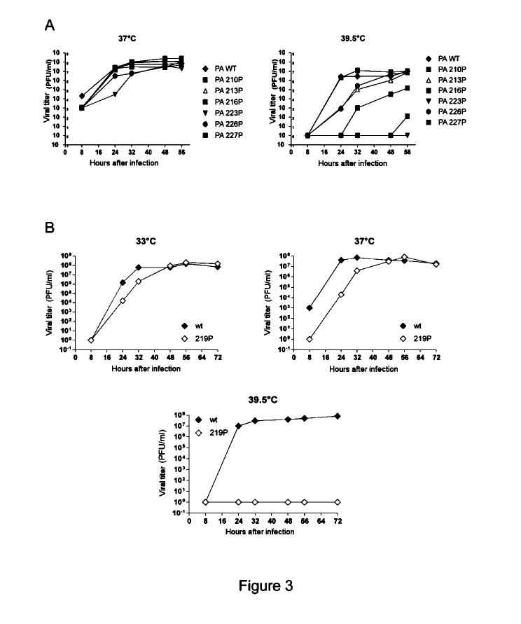

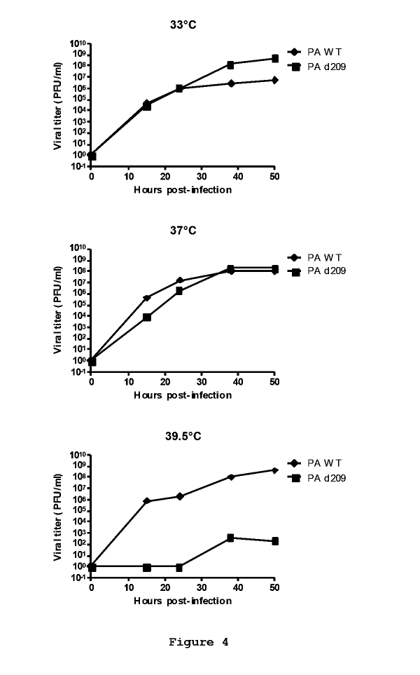

In order to better characterize the thermosensitivity of the mutants, viral replication tests on MDCK cells were carried out, at 37° C. and at 39.5° C. for the T210P, K213P, D216P, F223P, L226P and E227P viruses, and also at 33° C., 37° C. and 39.5° C. for the L219P and d209 viruses. The viral production (in plaque-forming units (PFU)/ml) was quantified at various times after infection (MOI 0.01). The results were illustrated by FIG. 3 (A: virus T210P, K213P, D216P, F223P, L226P and E227P viruses; B: L219P virus) and FIG. 4 (d209).

All the mutants have, at 37° C., replication kinetics similar to those of the wild-type strain WSN, with the exception of the F223P and L219P mutants, the replication of which appears to be slowed down. At 39.5° C., the replication is slowed down for all the mutants, with the exception of E227P, compared with the wild-type virus. This inhibition of replication appears to be moderate for K213P and L226P, for which, 56 hours after infection, a viral titer equivalent to that observed with the wild-type virus is observed. On the other hand, it is considerable for T210P, D216P and d209, and complete blockage of the replication is even observed in the case of the F223P and L219P mutants.

The effects of the mutations performed are summarized in table I below:

TABLE I

| | Viruses which could be | Thermo- |
|---|---|---|
| | obtained by reverse genetics | sensitivity |
| T210P | + | ++ |
| T210P 191FS | + | ++ |
| T210A | + | − |
| K213P | + | + |
| K213A | + | − |
| L214P | − | |
| L214A | + | − |
| D216P | + | +++ |
| D216P 191FS | + | +++ |
| D216A | + | − |
| Q217P | + | − |
| Q217A | + | − |
| S218P | − | |
| L219P | + | +++ |
| L219A | + | +++ |
| P220A | + | − |
| P221A | + | ++ |
| N222P | + | + |
| N222A | + | − |
| F223P | + | +++ |
| F223A | + | − |
| S224P | + | − |
| S225P | + | + |
| L226P | + | ++ |
| L226A | + | − |
| E227P | + | − |
| E227A | + | − |
| E237P | − | |
| E237A | + | − |
| T210P + | + | +++ |
| D216P | | |
| T210P + | + | +++ |
| D216P 191FS | | |
| d205 | − | |
| d206 | − | |
| d207 | + | +++ |
| d208 | + | ++ |
| d209 | + | ++ |
| d210 | + | ++ |
| d211 | − | |
| d212 | − | |

The thermosensitive phenotype appears to be much more marked in the case of the substitutions by a proline than in the case of the substitutions by an alanine. In addition, the thermosensitive phenotype can also be obtained with deletions of a codon.

### Example 2

### Polymerase Activity of the Replication Complexes Containing a Mutated PA Protein

In order to study the effects of the substitutions on the transcription of the viral RNAs, the activity of the mutated PA proteins was tested using a minireplicon system. The pPol1-WSN-NA-luciferase plasmid produces a modified viral RNA, in which the region encoding neuraminidase is replaced by the sequence encoding firefly luciferase. When this plasmid is cotransfected with plasmids expressing the PB1, PB2, NP and PA viral proteins, the luciferase production reflects the transcription and replication activity of the replication complex formed by these proteins.

48 hours after the cotransfection of the 293T cells, said cells were lysed, and the luciferase activity in the lysate was quantified.

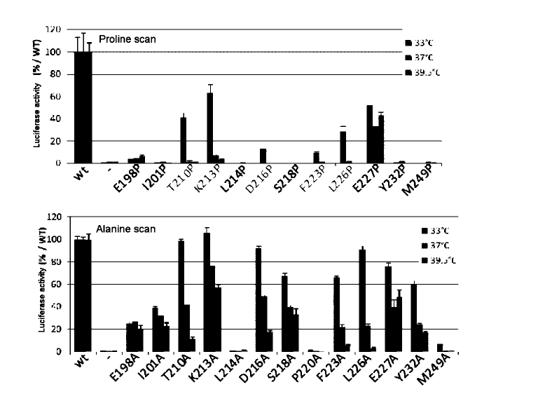

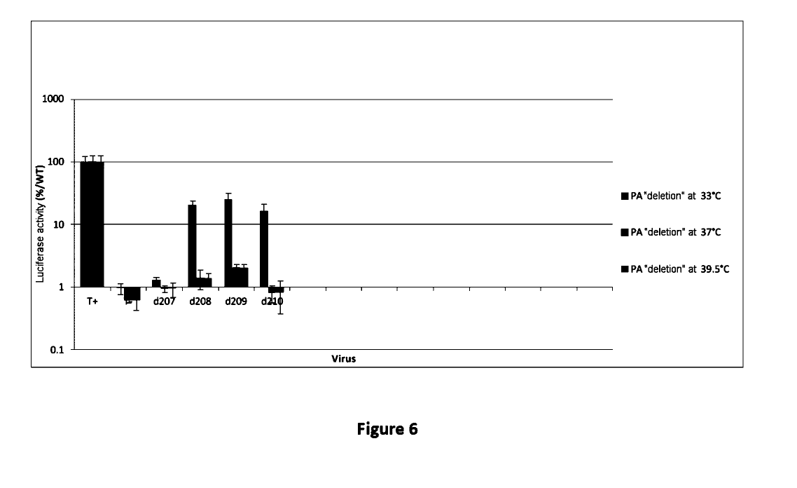

The results are illustrated by FIG. 5 and FIG. 6. These results represent the mean luciferase activity (+/− standard deviation), measured on three experiments, and standardized relative to the β-Galactosidase activity. FIG. 5: top panel: substitutions by a proline; bottom panel: substitution by an alanine.

The mutants carrying a substitution by a proline always show greater effects on the replicative capacity than their homologs substituted by an alanine. Almost all the mutations have less of an effect at 33° C. than at 37° C. and 39.5° C., thereby suggesting that the conformation of the hinge region of PA has an influence on the polymerase activity. Contrary to all the other mutants substituted by a proline, the E227P mutant shows a similar activity at 33° C., 37° C. and 39.5° C., in agreement with the absence of thermosensitivity of the virus carrying the same mutation.

All the mutations which induce a thermosensitive viral phenotype (210P, 213P, 216P, 221A, 222P, 223P, 226P, d207, d208, d209 and d210) show an effective polymerase activity at 33° C., and a weaker activity at 39.5° C., and even at 37° C. The d207 mutation induces a thermosensitive viral phenotype and shows a very weak polymerase activity at 33° C., and also at 37° C. and 39.5° C. Several variants (E198P, I201P, L214P, S218P, P220A, Y232P, M249P) do not show any or show little luciferase activity at 33° C., 37° C. and 39.5° C. These mutations probably lead to the loss of polymerase activity, inducing the replication defect observed in the corresponding mutant viruses. The P220A variant which does not have the thermosensitive viral phenotype could nevertheless be produced.

### Example 3

### Pathogenicity of the Mutant Viruses

The pathogenicity of the mutant viruses compared with the wild-type virus was evaluated on mice, after intranasal administration of the viruses tested, by monitoring the curve of weight and the prediction of mortality.

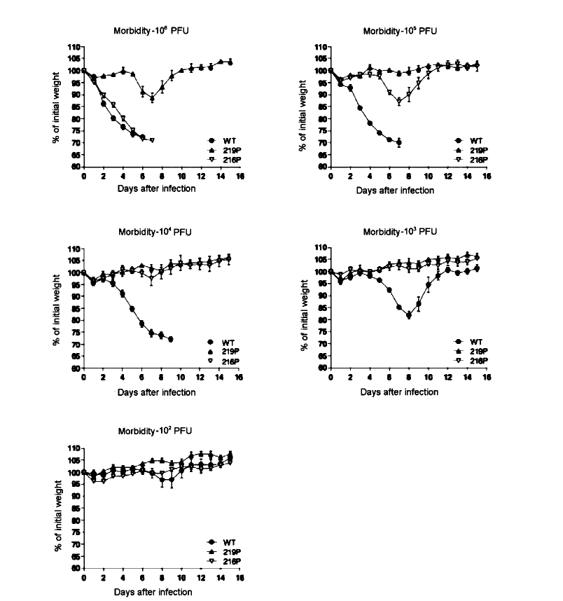

The results are illustrated by FIG. 7.

At doses of 10³ and 10⁴ plaque-forming units/mouse, the WSN virus induces a marked weight loss, whereas the two mutant viruses do not cause any weight loss. At a dose of 10⁵ plaque-forming units/mouse, while the WSN virus causes a rapid decrease in weight and the D216P mutant a less rapid and transient decrease in weight, the L219P mutant still has no effect on the weight of the infected animals. At 10⁶ plaque-forming units/mouse, the WSN and D216P viruses induce a rapid weight loss and the L219P mutant a transient weight loss.

The lethal dose 50 (LD50), based on the prediction of death or of survival of the animals, is estimated at 3.5 (in Log of plaque-forming units) for the wild-type virus, >6 for the L219P mutant, and 5.5 for D216P.

These results show that the thermosensitive phenotype is accompanied by an attenuation of the pathogenicity, which is particularly considerable in the case of the L219P virus.

### Example 4

### Expression of the Thermosensitivity Mutations in Other Strains of the Influenza Virus

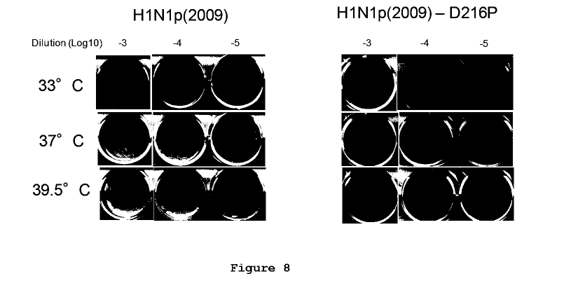

In order to determine whether the thermosensitive (Cs) phenotype of the mutations identified can be expressed in another genetic background, the D216P mutation was introduced into the PA genomic segment of the A(H1N1)pdm09 virus, using a previously described reverse genetics system (LeGoff et al., PLoS One, 2012, 7(8):e370952012). A mutant virus carrying the D216P mutation was produced (H1N1p(2009)-D216P). The replication properties in cell cultures of the mutant virus were examined by MDCK cell plaque titration. The results obtained are illustrated by FIG. 8. The D216P mutation induces a is phenotype in the A(H1N1)pdm09 virus. While the H1N1p(2009)-D216P virus does not lyse, or lyses only weakly, the MDCK cells at 37° C. and 39.5° C., it has a lytic efficiency comparable to that of the wild-type H1N1p(2009) virus at 33° C. These results demonstrate that this ts characteristic can be expressed on strains other than the WSN virus.

### Example 5

### Induction of Anti-Influenza Antibodies by the Mutant Viruses

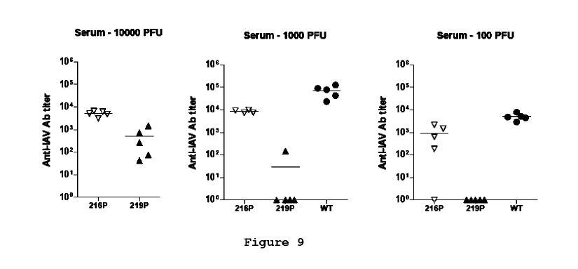

The induction of an adaptive immune response by the mutant PA viruses was analyzed in the surviving mice of example 3, two weeks and four days after infection. The anti-influenza antibody titers induced by the mutant PA viruses were measured using a conventional ELISA assay, so as to evaluate the potential of these mutants to behave like effective live vaccines. The results are illustrated by FIG. 9. At an infectious dose of 10² or 10³ PFU, the D216P mutant and the wild-type virus, but not the L219P mutant, induce a strong antibody response, suggesting that the thermosensitivity of the L219P mutant is too marked to replicate and induce an effective immune response. At an infectious dose of 10⁴ PFU, the L219P mutant is capable of inducing antibody synthesis. These results indicate that the two thermosensitive mutants D216P and L219P are strongly attenuated and capable of inducing an effective antibody response.

### Example 6

### Correlation Between the Thermosensitive Phenotype of the Influenza Mutants and the Introduction of a Mutation into a Flexible Domain of an Influenza Protein

The correlation between the introduction of a mutation into a flexible zone of an influenza protein and the obtaining of a thermosensitive mutant virus was studied on two different influenza proteins, PA and NP.

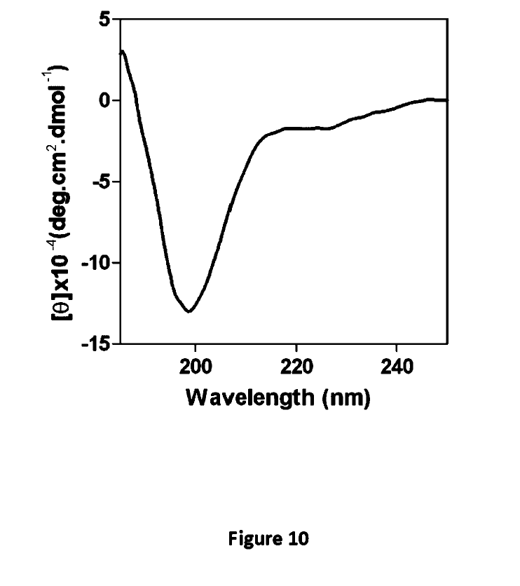

The hinge region of the PA protein of the influenza A/WSN/1933(H1N1) virus (amino acids 197 to 256) was expressed in E. coli and purified to homogeneity, according to the standard protocols for expressing and purifying recombinant proteins in E. coli. The secondary structure of this region was analyzed by circular dichroism. The spectrum obtained shows the absence of a stable secondary structure on this segment (FIG. 10). These results indicate that the hinge region of the PA protein which makes it possible to obtain thermosensitive viruses corresponds to a flexible domain of the influenza PA protein.

Flexible domains of the NP protein were identified from the atomic structure of the NP protein of WSN, previously characterized (Ye et al., Nature, 444, 1078-82, 2006). NP domains appeared non-structured in the crystal: the positions of residues 73 to 92, 203 to 212, 230 and 231, 297 to 301, 429 to 436 and 490 to 498 are not defined. Substitutions for alanine codons were carried out on residues 203, 204, 205, 206, 207, 209, 210 and 211, as described in example 1. Mutant viruses could be isolated for the 205, 206, 209, 210 and 211 mutations. The D203A, R204A and W207A mutant viruses could not be produced.

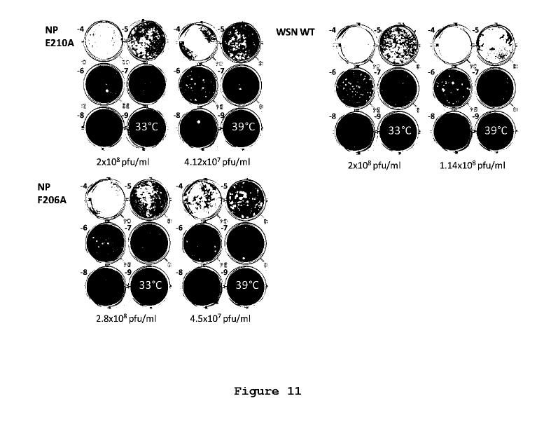

The replication properties in cell cultures of the mutant viruses which could be obtained were examined by MDCK cell plaque titration. Two mutants, F206A and E210A, have a is phenotype. The results are illustrated by FIG. 11. The F206A and E210A mutations induce a is phenotype in the WSN virus. Although the NP-F206A and NP-E2101A viruses do not lyse, or lyse only weakly, the MDCK cells at 39.5° C., they have a lytic efficiency comparable to that of the WN wild-type virus at 33° C.

These results indicate that the flexible domains of the influenza proteins represent preferential target domains for obtaining thermosensitive mutant viruses.

1. A method for preparing a thermosensitive mutant of an influenza virus, characterized in that at least one synthetic mutation is introduced into a region of a gene encoding the PA, PB1, PB2, NP, HA, HE, NA, M1, M2, NS1 or NS2 protein of said virus, said mutation being located in a region of said gene corresponding to a flexible region of said protein and generating a thermosensitive mutant of said virus.

2. The method as claimed in claim 1, characterized in that said mutation is in a region of a gene encoding the PA, PB1, PB2 or NP protein.

3. The method as claimed in claim 2, characterized in that said mutation is located in the region of the gene encoding the PA protein corresponding to region 197-257 of the PA protein of the influenza A virus.

4. The method as claimed in claim 3, characterized in that said mutation is located in the region of said gene corresponding to region 197-225 of the PA protein of the influenza A virus.

5. The method as claimed in claim 4, characterized in that said mutation induces the deletion of at least one of the amino acids corresponding to positions 207, 208, 209 or 210 of the PA protein of the influenza A virus.

6. The method as claimed in claim 4, characterized in that said mutation is chosen from:
a mutation resulting in the substitution of the amino acid corresponding to position 216 of the PA subunit of influenza A, by an amino acid other than an asparagine or an aspartic acid;
a mutation resulting in the substitution of the amino acid corresponding to position 219 of the PA subunit of influenza A, by an amino acid other than a leucine or a valine;
a mutation resulting in the substitution of the amino acid corresponding to position 210 of the PA subunit of influenza A, by an amino acid other than a threonine;
a mutation resulting in the substitution of the amino acid corresponding to position 213 of the PA subunit of influenza A, by an amino acid other than an arginine or a lysine;
a mutation resulting in the substitution of the amino acid corresponding to position 221 of the PA subunit of influenza A, by an amino acid other than a proline in the case of influenza A, an alanine in the case of influenza B, and a leucine in the case of influenza C;
a mutation resulting in the substitution of the amino acid corresponding to position 222 of the PA subunit of influenza A, by an amino acid other than an asparagine in the case of influenza A, a glycine in the case of influenza B, and a proline in the case of influenza C;
a mutation resulting in the substitution of the amino acid corresponding to position 223 of the PA subunit of influenza A, by an amino acid other than a phenylalanine; and
a mutation resulting in the substitution of the amino acid corresponding to position 225 of the PA subunit of influenza A, by an amino acid other than a serine in the case of influenza A, an asparagine in the case of influenza B, and a threonine in the case of influenza C.

7. The method as claimed in claim 6, characterized in that said mutation includes the substitution of at least one of the amino acids corresponding to positions 216 or 219 of said PA subunit in influenza A by an alanine or a proline.

8. The method as claimed in claim 2, characterized in that said mutation is located in the region of the gene encoding the NP protein corresponding to region 73 to 92, 203 to 212, 230 and 231, 297 to 301, 429 to 436 or 490 to 498 of the NP protein of the influenza A virus.

9. The method as claimed in claim 8, characterized in that said mutation induces the deletion or the substitution of at least one of the amino acids corresponding to position 206 or 210 of the NP protein of the influenza A virus.

10. The method as claimed in claim 1, characterized in that several of said mutations are introduced, said mutations inducing the substitution or the deletion of an amino acid and being located in one or more regions of one or more genes of said influenza virus.

11. A mutant recombinant influenza virus characterized in that a gene of said virus contains at least one mutation as defined in claim 1.

12. The mutant recombinant influenza virus as claimed in claim 11, as a live attenuated flu vaccine.
